(19) United States
(12) Patent Application Publication
(10) Pub. No.: US 2003/0114919 A1
(43) Pub. Date: Jun. 19, 2003

(54) Polymeric stent with metallic rings

(76) Inventors: Jesse McQuiston, Terre Haute, IN (US); Santosh Prabhu, San Jose, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/014,052

(22) Filed: Dec. 10, 2001

(51) Int. Cl. A61F 2/06

(52) U.S. Cl. 623/1.15

(57) ABSTRACT

An expandable stent for implantation in a body lumen, such as a coronary artery, consists of radially expandable cylindrical rings generally aligned on a common axis and disposed around a polymeric tube. The polymeric tube provides longitudinal and flexural flexibility to facilitate delivery through tortuous body lumens and the rings provide the necessary radial strength to maintain the patency of a vessel and to resist collapse.

### BACKGROUND OF THE INVENTION

This invention relates to expandable endoprosthesis devices, generally called stents, which are adapted to be implanted into a patient's body lumen, such as blood vessel, to maintain the patency thereof. These devices are useful in the treatment of atherosclerotic stenosis in blood vessels.

Stents are generally tubular-shaped devices which function to hold open a segment of a blood vessel, coronary artery, or other anatomical lumen. They are particularly suitable for use to support and hold back a dissected arterial lining which can occlude the fluid passageway therethrough.

Various means have been described to deliver and implant stents. One method frequently described for delivering a stent to a desired intraluminal location includes mounting the expandable stent on an expandable member, such as a balloon, provided on the distal end of an intravascular catheter, advancing the catheter to the desired location within the patient's body lumen, inflating the balloon on the catheter to expand the stent into a permanent expanded condition and then deflating the balloon and removing the catheter. One of the difficulties encountered using prior stents involved maintaining the radial rigidity needed to hold open a body lumen while at the same time maintaining the longitudinal flexibility of the stent to facilitate its delivery. Once the stent is mounted on the balloon portion of the catheter, it is often delivered through tortuous vessels, including tortuous coronary arteries. The stent must have numerous properties and characteristics, including a high degree of flexibility in order to appropriately navigate the tortuous coronary arteries. This flexibility must be balanced against other features including radial strength once the stent has been expanded and implanted in the artery. While other numerous prior art stents have had sufficient radial strength to hold open and maintain the patency of a coronary artery, they have lacked the flexibility required to easily navigate tortuous vessels without damaging the vessels during delivery.

Generally speaking, most prior art intravascular stents are formed from a metal such as stainless steel, which is balloon expandable and plastically deforms upon expansion to hold open a vessel. The component parts of these types of stents typically are all formed of the same type of metal, i.e., stainless steel. Other types of prior art stents may be formed from a polymer, again all of the component parts being formed from the same polymer material. These types of stents, the ones formed from a metal and the ones formed from a polymer, each have advantages and disadvantages. One of the advantages of the metallic stents is their high radial strength once expanded and implanted in the vessel. A disadvantage may be that the metallic stent lacks flexibility which is important during the delivery of the stent to the target site. With respect to polymer stents, they may have a tendency to be quite flexible and are advantageous for use during delivery through tortuous vessels, however, such polymer stents may lack the radial strength necessary to adequately support the lumen once implanted.

What has been needed and heretofore unavailable is a stent which has a high degree of flexibility so that it can be advanced through tortuous passageways and can be readily expanded and yet have the mechanical strength to hold open the body lumen into which it expanded. The present invention satisfied this need.

### SUMMARY OF THE INVENTION

The present invention is directed to an expandable stent for implantation in a body lumen, such as a coronary artery. The stent consists of radially expandable cylindrical rings generally aligned on a common axis along a polymeric tube. The polymeric tube provides longitudinal and flexural flexibility to facilitate delivery through tortuous body lumens and the rings maintain sufficient radial strength to maintain the patency of a vessel and to resist collapse.

The stent of the present invention generally includes apolymeric tube and a plurality of flexible metallic radially expandable cylindrical rings, the rings relatively independent in their ability to expand and to flex relative to one another. The individual radially expandable cylindrical rings of the stent are formed from a metallic material and are aligned on a common longitudinal axis along the tube. The cylindrical rings can be formed with undulations having peaks and valleys generally formed as U, W, and Y members. The peaks of each cylindrical ring can be axially aligned with the peaks and valleys of each adjacent cylindrical ring to provide the desired flexibility. The resulting stent structure is a series of radially expandable cylindrical rings which are spaced longitudinally close enough so that small dissections in the wall of a body lumen may be pressed back into position against the lumenal wall, but not so close as to compromise the longitudinal flexibility of the stent.

The cylindrical rings are placed over the polymeric tube and attached at predetermined locations on the tube. The cylindrical rings and polymeric tube can be connected in a number of ways not limited to a slotted fitting, a bonding agent, and an interference fit. The polymeric tube material provides flexibility and allows the stent to easily bend or flex along its longitudinal axis as the stent navigates through tortuous vessels or coronary arteries. The combination of the flexible metallic cylindrical rings and the polymeric tube produces a stent which is flexible along its length and about its longitudinal axis, yet maintains stiffness in the radial direction after it has been expanded. The stiffness of the stent helps to resist collapse and maintain the patency of the vessel.

The polymeric tube can be formed with a mesh pattern to enable the stent to have much higher flexibility and deliverability than traditional all-metal stents. For example, when used in conjunction with the metallic cylindrical rings the polymeric mesh can be configured to have 200% less resistance to longitudinal bending in one form and 200% more resistance to compression when compared with a similarly sized all metal stent in another form. The radial strength can benefit by the addition of more metallic cylindrical rings than a conventional all metal stent. With the polymeric mesh, the addition of metallic cylindrical rings does not significantly compromise flexibility as it would in a all metal stent, the result being greater resistance to compression than all metal stents.

The mesh pattern can be configured with a more packed cell structure than all metal stents without compromising flexibility. Upon expansion the stent will conform well to the arterial walls due to the presence of the packed cell structure. This packed cell structure will provide better scaffolding and minimize the chances of plaque prolapse.

The stent of the present invention can also be used as a platform for local drug delivery. Lack of uniformity of drug distribution to the arterial walls is one of the main drawbacks of the current drug delivery stents. The polymeric tube of the stent can be loaded with anti-restenotic drugs and because it has a more packed cell structure than conventional all metal stents, the delivery of the drug will be more uniform to the arterial walls.

The stent embodying features of the invention can be readily delivered to the desired body lumen, such as a coronary artery (peripheral vessels, bile ducts, etc.), by mounting the stent on an expandable member of a delivery catheter, for example a balloon, and advancing the catheter and stent assembly through the body lumen to the target site. Generally, a crimping tool is used to crimp the stent onto the balloon portion of the catheter so that the stent does not move longitudinally relative to the balloon portion of the catheter during delivery through the arteries, and during expansion of the stent at the target site.

During the crimping process the metallic cylindrical rings undergo a plastic deformation and radially compress while the polymeric tube also radially compresses within the rings to removably secure the stent to the balloon.

After insertion of the stent to the desired location of delivery, the balloon is inflated to implant the stent. During expansion of the stent, portions of the cylindrical rings may tip outwardly resulting in projecting members on the outer surface of the expanded stent. These projecting members tip radially outwardly from the outer surface of the stent and embed into the vessel wall and help secure the expanded stent so that it does not move once it is implanted.

It is to be recognized that the stent of the present invention can be self-expanding or balloon-expanded. Moreover, the present invention can be modified to be used in other body lumens including highly tortuous and distal vasculature as well as to create whole or portions of other medical devices or markers placed on such devices.

Other features and advantages of the present invention will become more apparent from the following detailed description of the invention when taken in conjunction with the accompanying exemplary drawings.

### BRIEF DESCRIPTION OF THE DRAWINGS

FIG. 1 is an elevational view, partially in section, of a stent embodying features of the invention which is mounted on a delivery catheter and disposed within an artery.

FIG. 2 is an elevational view, partially in section, similar to that shown in FIG. 1 wherein the stent is expanded within an artery.

FIG. 3 is an elevational view, partially in section, depicting the expanded stent within the artery after withdrawal of the delivery catheter.

FIG. 4 is a plan view of a flattened section of the stent of the invention, illustrating the cylindrical rings attached to the polymeric tube in the crimped state.

FIG. 5 is a perspective view of the stent of FIG. 4 after it is fully expanded depicting some portions of the stent projecting radially outwardly.

FIG. 6 is a perspective view of a mandrel having grooves for the polymeric mesh for use in the injection molding process.

FIG. 7 is a cross-sectional view of a section of the mandrel having grooves for the polymeric mesh.

FIG. 8 is a perspective view of a quarter arc section of the outer mold cover having grooves for the polymeric mesh.

FIG. 9 is a perspective view of the mandrel with four quarter arc section outer mold covers positioned over the mandrel for use in the injection molding process.

FIG. 10 is a perspective view of the stent of the invention positioned on the mandrel and illustrating the cylindrical rings disposed around the polymeric mesh after the injection molding process.

### DETAILED DESCRIPTION OF THE PREFERRED EMBODIMENTS

The present invention improves on existing stents by providing a combination of a polymeric tube and a series of metallic cylindrical rings that combine to form a more flexible hybrid stent without sacrificing radial strength. The metallic cylindrical rings are longitudinally aligned along an axis of the stent and disposed over the polymeric tube.

FIG. 1 illustrates a stent 10 incorporating features of the invention which is mounted onto a delivery catheter 11. The stent generally comprises a plurality of radially expandable cylindrical rings 12 disposed generally coaxially and bonded to the polymeric tube 13. The delivery catheter 11 has an expandable portion or balloon 14 for expanding of the stent 10 within an artery 15. The artery 15, as shown in FIG. 1 has an occluded portion of the arterial passageway that has been opened by a previous procedure, such as angioplasty.

The delivery catheter 11 onto which the stent 10 is mounted, is essentially the same as a conventional balloon dilatation catheter for angioplasty procedures. The balloon 14 may be formed of suitable materials such as polyethylene, polyethylene terephthalate, polyvinyl chloride, nylon and ionomers such as Surlyn® manufactured by the Polymer Products Division of the Du Pont Company. Other polymers may also be used. In order for the stent 10 to remain in place on the balloon 14 during delivery to the site of the damage within the artery 15, the stent 10 is crimped or compressed onto the balloon in a known manner. FIG. 1 shows the stent 10 in its crimped state with the polymeric tube 13 and the cylindrical rings 12 in radially compressed forms.

Each radially expandable cylindrical ring 12 of the stent 10 may be substantially independently expanded to some degree relative to adjacent rings and therefore can have a tapered configuration. Similarly, the balloon 14 may be provided with an inflated shape other than cylindrical, e.g., tapered, to facilitate implantation of the tapered stent in a variety of body lumen shapes.

In one embodiment, the delivery of the stent 10 is accomplished in the following manner. The stent is first mounted onto the inflatable balloon 14 on the distal extremity of the delivery catheter by crimping or compressing the stent in a known manner. During the crimping process the metallic rings 12 undergo a plastic deformation and are responsible for securing the stent to the balloon. The rings also are responsible for holding the polymeric tube 13 in a compressed state.

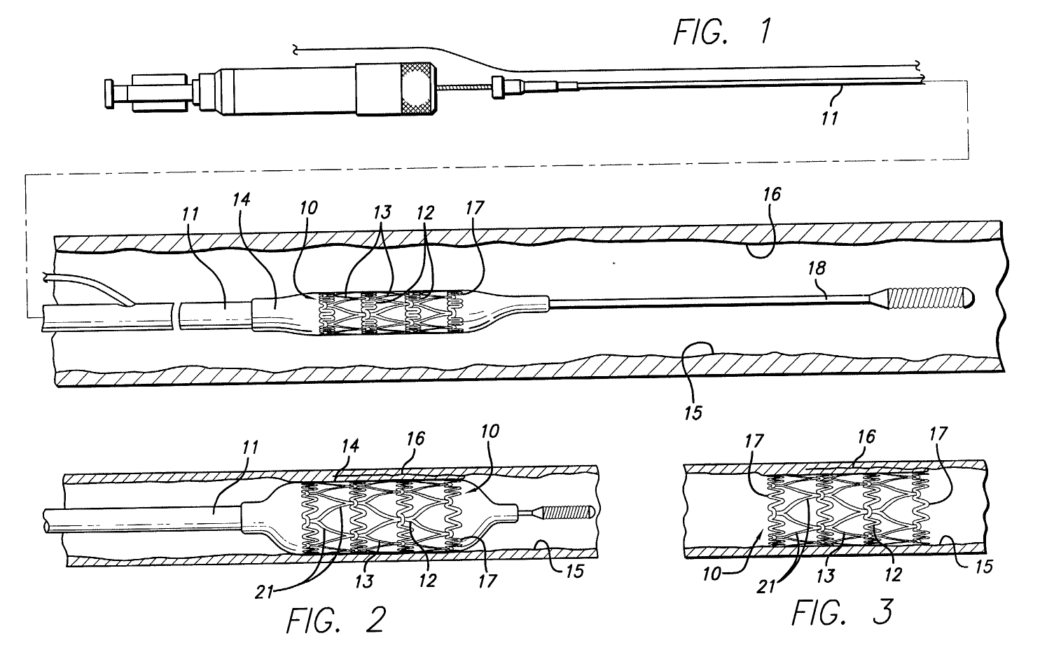

The catheter-stent assembly is introduced within the patient's vasculature in a conventional Seldinger technique through a guiding catheter (not shown). A guide wire 18 is disposed across the damaged arterial section and then the catheter-stent assembly is advanced over a guide wire 18 within the artery 15 until the stent is positioned at the target site 16. The balloon of the catheter is expanded, expanding the stent against the artery, which is illustrated in FIG. 2. During expansion the diameter of the cylindrical rings 12 and the polymeric tube 13 increase at substantially the same rate. The similar rate of expansion helps keep the cylindrical rings and polymeric tube closely coupled together. While not shown in the drawing, the artery is preferably expanded slightly by the expansion of the stent to seat or otherwise fix the stent to prevent movement. In some circumstances during the treatment of stenotic portions of an artery, the artery may have to be expanded considerably in order to facilitate passage of blood or other fluid therethrough.

The stent may also be self-expanding and include metallic cylindrical rings 12 made from a shape memory alloy which is a superelastic material such as nickel titanium. Along the same lines, the polymeric tube 13 can embody shape memory characteristics so that the tube could also be self-expanding.

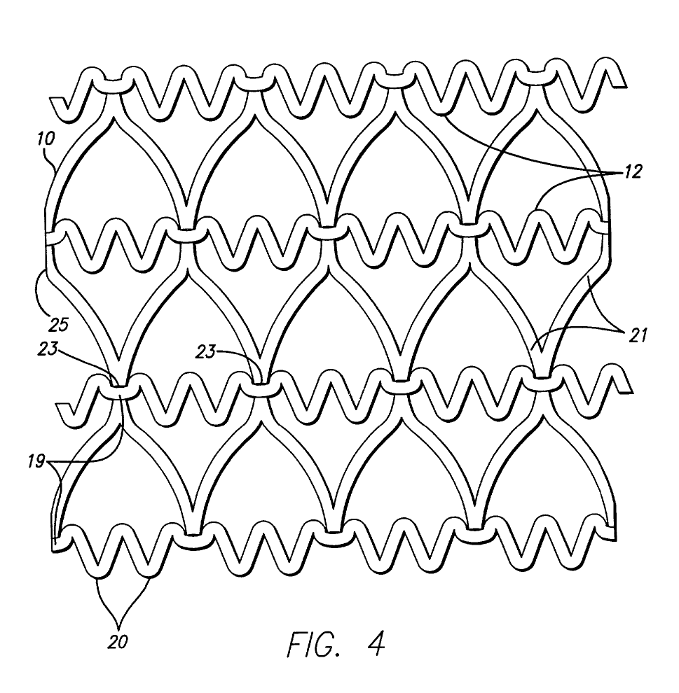

The stent 10 serves to hold open the artery 15 after the catheter 11 is withdrawn, as illustrated by FIG. 3. In the stent's expanded state, the polymeric tube 13 and the cylindrical rings 12 are in radially expanded forms. Due to the formation of the cylindrical rings 12 from an elongated tubular member or a flat sheet, the undulating component of the cylindrical rings 12 is relatively flat in transverse cross-section so that when the stent is expanded, the cylindrical rings are pressed into the wall of the artery and as a result do not interfere with the blood flow through the artery. The cylindrical rings will eventually be covered with endothelial cell growth which further minimizes blood flow interference and provide good tacking characteristics to prevent stent movement within the artery. Furthermore, the cylindrical rings 12 are aligned along the longitudinal axis and spaced at regular intervals to provide uniform support for the wall of the artery 15, and consequently are well adapted to tack up and hold in place small flaps or dissections in the wall of the artery 15.

The properties of the stent 10 may vary by alteration of the cylindrical rings 12. FIG. 4 illustrates a plain view of a flattened section of the stent in its crimped state. The cylindrical rings have an undulating shape including peaks and valleys formed as W-shaped members 20 which are out of phase with adjacent cylindrical rings. The particular pattern and how many undulations, or the amplitude of the undulations, are chosen to fill particular mechanical requirements for the stent, such as radial stiffness. The number of cylindrical rings 12 incorporated into the stent can also vary according to design requirements such as radial stiffness and longitudinal flexibility.

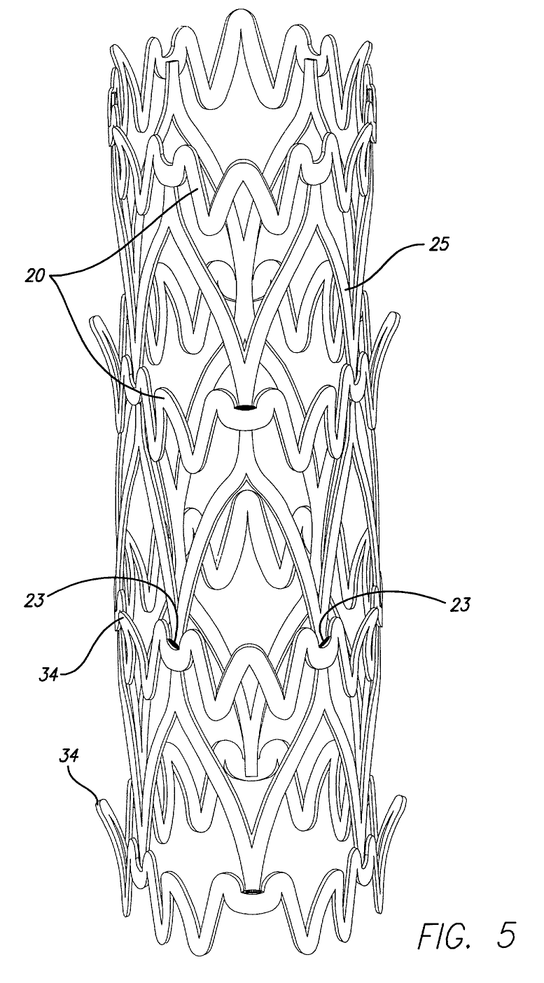

The W-shaped members 20 have a radius that evenly distributes expansion forces over the various peaks and valleys. After the cylindrical rings 12 have been radially expanded as shown in FIG. 5, outwardly projecting edges 34 are formed from the W-shaped members 20. That is, during radial expansion some of the W-shaped members may tip radially outwardly thereby forming outwardly projecting edges. These outwardly projecting edges 34 provide for a roughened outer wall surface of the stent 10 and assist in implanting the stent in the vascular wall by embedding into the vascular wall. In other words, outwardly projecting edges embed into the vascular wall, for example artery 15, as depicted in FIG. 3.

The stent patterns shown in FIGS. 1-5 are for illustration purposes only and can vary in shape and size to accommodate different vessels or body lumens. The cylindrical rings can have any structural shape not limited to the aforedescribed W-shaped members. For example, the cylindrical rings can also include U and Y-shaped members and a plethora of other shapes including generally Z-shapes, sine waves, loops, and sharp angles, according to design requirements. The cylindrical rings can also be formed with shape memory alloys, and radiopacitly enhanced.

In keeping with the invention, the polymeric tube 13 is formed from a flexible polymeric material, that is bendable and flexible to enhance longitudinal and flexural flexibility of the stent 10. The polymeric tube can be formed with a mesh pattern 21 to enable the stent to have higher flexibility and deliveribility than traditional all metal stents. The mesh pattern shown in FIG. 4 can generally be viewed as a plurality of oval-shaped members 25. The mesh can also be formed in a plethora of different patterns according to design requirements. For example, the mesh can be formed with more or less surface area, a greater or lower number of oval-shaped members, and a variety of other shapes incorporating generally U-, Y-, W-, and Z-shaped members along with sine waves, loops, and sharp angles.

The polymeric tube 13 with the mesh pattern 21 when used in connection with the metallic cylindrical rings 12 enables the stent 10 to have higher flexibility and deliverability than all metal stents. Referring to FIG. 4, the cylindrical rings 12 are formed out of a metal, such as stainless steel and can be attached with a bonding agent to the outer surface of the meshed polymeric tube 13. In this embodiment, the cylindrical rings 12 overlap the meshed polymeric tube 13. The amount of overlap can vary according to design requirements. For example, it is possible for less than 20% of the metal constituting the cylindrical rings 12 to be overlapped with less than 15% the polymer constituting the polymeric mesh. By comparison, an all metal stent generally consists of a series of metallic cylindrical rings interconnected by metallic links or struts. In the case of metallic stents where the rings and links are laser cut from a unitary thin-walled tube, the design of the stent is a compromise between flexibility and rigidity. The stent must be flexible enough to conform to the curvature of the body lumen it is inserted into and the stent must be rigid enough to remain in its expanded state once implanted.

In the stent of the present invention, the polymeric tube 13 is configured to enable the stent 10 to exceed the longitudinal flexibility of conventional metallic stents. Such flexibility is achieved by factors such as choice of polymeric material, type of mesh pattern, and desired final size. To account for the required radial strength, the cylindrical rings 12 are formed from a metallic material as in conventional metallic stents. Because of the relatively small longitudinal length of the metallic rings and because the rings are not connected with metallic links, the flexibility provided by the polymeric mesh is not significantly inhibited. Accordingly, it is possible with the present invention to produce a stent having radial strength equivalent to a conventional metallic stent while offering longitudinal flexibility exceeding the metallic stent. With the addition of more rings to the stent 10, radial stiffness can also be increased over a conventional stent while maintaining a high degree of flexibility.

The stent 10 may also be used in connection with a therapeutic agent to perform a variety of functions, from preventing blood clots to promoting healing. When compared with conventional all metal stents, the packed cell structure of the stent of the present invention enables the delivery of the drug to the arterial walls to be more uniform. The lack of uniformity of drug distribution to the arterial walls is one of the main drawbacks of the current metallic drug delivery stents.

As an example, an active agent loaded into or coated on the polymeric tube 13 can inhibit the activity of vascular smooth muscle cells. Similarly, an active agent coated on the cylindrical rings 12 can also inhibit the activity of vascular smooth muscle cells. More specifically, the active agent is aimed at inhibiting abnormal or inappropriate migration and proliferation of smooth muscle cells. The active agent can also include any substance capable of exerting a therapeutic or prophylactic effect in the practice of the present invention. The agent can also be for enhancing wound healing in a vascular site or improving the structural and elastic properties of the vascular site. The dosage or concentration of the active agent required to produce a favorable therapeutic effect should be less than the level at which the active agent produces toxic effects and greater than the level at which non-therapeutic results are obtained. The dosage or concentration of the active agent required to inhibit the desired cellular activity of the vascular region can depend upon factors such as the particular circumstances of the patient; the nature of the trauma; the nature of the therapy desired; the time over which the ingredient administered resides at the vascular site; and if other therapeutic agents are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

Examples of therapeutic agents include rapamycin, actinomycin D (ActD), or derivatives and analogs thereof ActD is manufactured by Sigma-Aldrich, 1001 West Saint Paul Avenue, Milwaukee Wis. 53233, or COSMEGEN, available from Merck. Synonyms of actinopmycin D include dactinomycin, actinomycin IV, actinomycin l1, actinomycin X1, and actinomycin C1. Examples of agents include other antiproliferative substances as well as antineoplastic, antinflammatory, antiplatelet, anticoagulant, antifibrin, antithomobin, antimitotic, antibiotic, and antioxidant substances. Examples of antineoplastics include taxol (paclitaxel and docetaxel). Examples of antiplatelets,, anticoagulants, antifibrins, and antithrombins include sodium heparin, low molecular weight heparin, hirudin, argatroban, forskolin, vapiprost, prostacyclin and prostacyclin analogs, dextran, D-phe-pro-arg-chloromethylketone (synthetic antithrombin), dipyridamole, glycoprotein, 11b/111a platelet membrane receptor antagonist, recombinant hirudin, thrombin inhibitor (available from Biogen), and 7E-3B® (an antiplatelet drug from Centocore). Examples of antimitotic agents include methotrexate, azathioprine, vincristine, vinbiastine, fluorouracil, adriamycin, and mutamycin. Examples of cytostatic or antiproliferative agents include angiopeptin (a somatostatin analog from Ibsen), angiotensin converting enzyme inhibitors such as Captopril (available from Squibb), Cilazapril (available from Hoffman-LaRoche), or Lisinopril (available from Merck); calcium channel blockers (such as Nifedipine), colchicine fibroblast growth factor (FGF) antagonists, fish oil (omega 3-fatty acid), histamine antagonist, Lovastatin (an inhibitor of HMG-CoA reductase, a cholesterol lowering drug from Merck), monoclonal antibodies (such as PDGF receptors), nitroprusside, phosphodiesterase inhibitors, prostaglandin inhibitor (available from Glazo), Seramin (a PDGF antagonist), serotonin blockers, steroids, thioprotease inhibitors, triazolopyrimidine (a PDGF antagonist), and nitric oxide. Other therapeutic substances or agents which may be appropriate include alpha-interferon, genetically engineered epithelial cells, and dexamethasone.

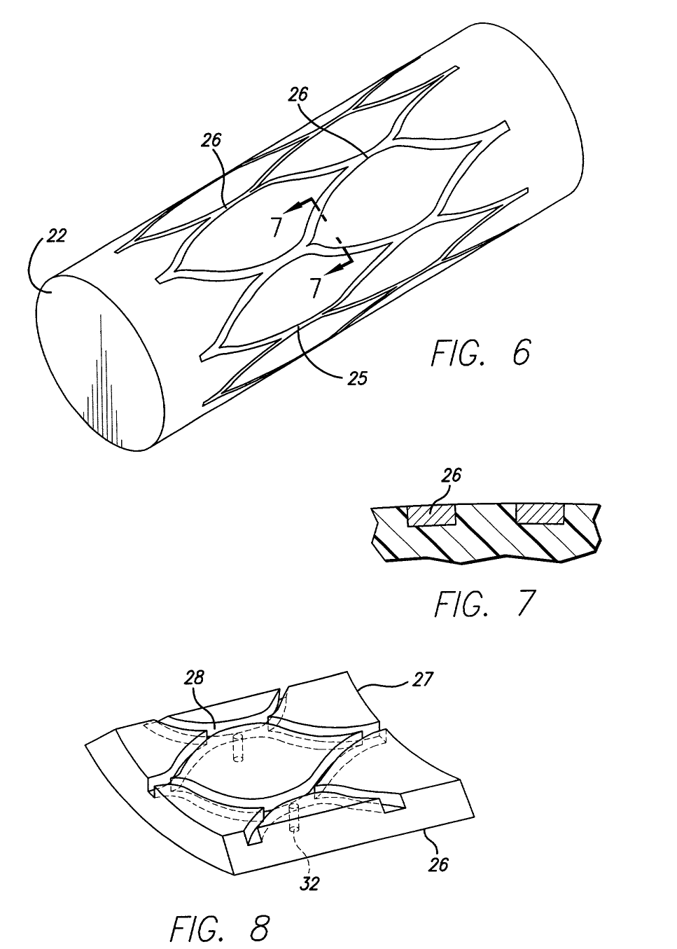

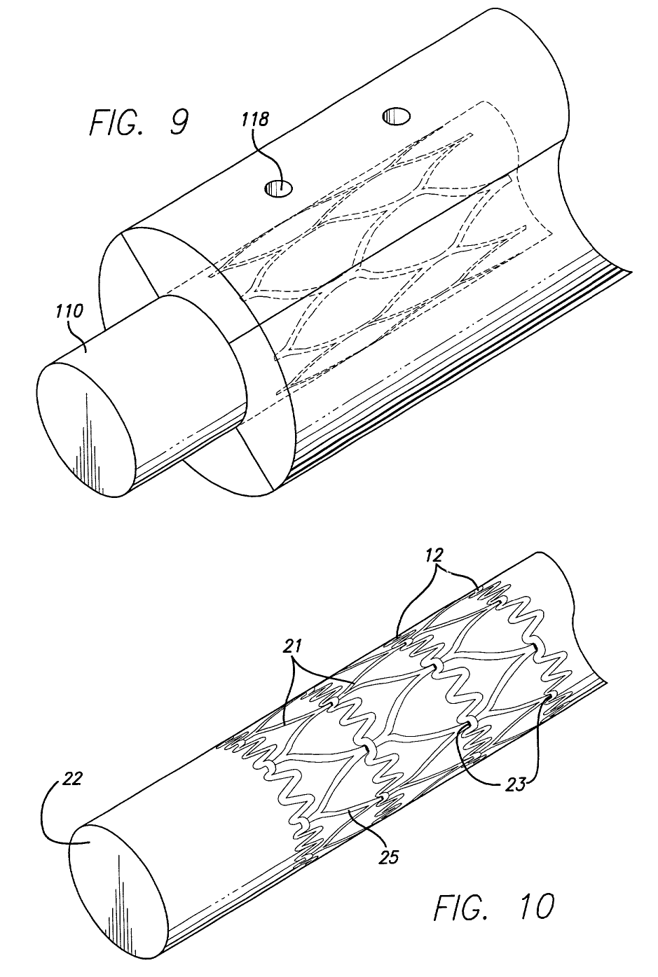

One method of making the stent 10 of the invention is to first form the polymeric tube 13 by injection molding. An injection molding apparatus is shown in FIGS. 6-9. In keeping with the invention, a mandrel 22 is provided with mesh grooves 26 that correspond to the pattern of the polymeric mesh. The outer mold covers 27 typically are in cylindrical sections as depicted in FIGS. 8 and 9 and it is preferred that from two to four arc sections of outer mold covers be used to encase the mandrel 22. Each of the outer mold covers has outer mesh grooves 28 that correspond to mesh grooves 26 in the mandrel.

The mandrel and the encapsulating sleeve permit the injection of a polymer which fills the channels corresponding to the mesh pattern. The polymer used to form the mesh is injected by known techniques through gates 32 located at multiple positions along the outer mold covers. The gates provide openings or apertures through the outer mold coves to correspond to the location of the mesh grooves 26, 28 so that as the polymer is injected through the outer mold cover, it will flow into the mesh grooves 26, 28 and form the mesh pattern.

For example, referring to FIGS. 6-9, the outer mold covers 27 are placed over the mandrel so that the mold cover outer mesh grooves 28 correspond to the mandrel mesh grooves 26. After the polymer material is injected through gates 32, the assembly is allowed to cool and the outer mold covers are removed from the mandrel 22 and any excess flashing from the gates 32 can be removed by known means.

The polymeric tubing 13 may also be formed by laser cutting a flat polymeric sheet in the form of the mesh pattern 21, and then rolling the pattern into the shape of the cylindrical tube and providing a longitudinal bond to form the stent. Other methods of forming the polymeric tube are well known and include coiling a polymeric wire to form the tube, injection molding of a thermoplastic as mentioned above and reaction injection molding of a thermoset polymeric material.

The polymeric tube 13 can be made from polyurethanes, polyolefins, polyesters, polyamides, flouropolymers and their co-polymers, polyetherurethanes, polyesterurethanes, silicone, thermoplastic elastomer (e.g., C-flex), polyether-amide thermoplastic elastomer (e.g., Pebax), fluoroelastomers, fluorosilicone elastomer, styrene-butadiene-styrene rubber, styrene-isoprene-styrene rubber, polyisoprene, neoprene (polychloroprene), ploybutadiene, ethylene-propylene elastomer, chlorosulfonated polyethylene elastomer, butyl rubber, polysulfide elastomer, polyacrylate elastomer, nitrile rubber, a family of elastomers composed of styrene, ethylene, propylene, aliphatic polyearbonate polyurethane, polymers augmented with antioxidents, polymers augmented with image enhancing materials, polymers having a proton (H+) core, polymers augmented with protons (H+), butadiene and isoprene (e.g., Kraton) andpolyesterthermoplastic elastomer (e.g., Hytrel). The polymeric tube can also be made from a shape memory polymer, be radiopacity enhanced and incorporate a material that generates a magnetic susceptibility artifact of the stent.

One method of making the rings is to laser cut the cylindrical rings 12 from a thin-walled tubular member, such as stainless steel tubing to remove portions of the tubing in the desired pattern for the rings, leaving relatively untouched the portions of the metallic tubing which are to form the rings. In accordance with the invention, it is preferred to cut the tubing in the desired pattern by means of a machine-controlled laser as is well known in the art.

The cylindrical rings 12 can be made from stainless steel, titanium, tantalum, nickel titanium, cobalt-chromium, gold, paladium, platinum and iridium. In the case of a suitable biocompatible material such as stainless steel, the stainless steel tube may be Alloy type: 316L SS, Special Chemistry per ASTM F138-92 or ASTM F139-92 grade 2. Special Chemistry of type 316L per ASTM F138-92 or ASTM F139-92 Stainless Steel for Surgical Implants in weight percent.

| | |
|---|---|
| Carbon (C) | 0.03% max. |
| Manganese (Mn) | 2.00% max. |
| Phosphorous (P) | 0.025% max. |
| Sulphur (S) | 0.010% max. |
| Silicon (Si) | 0.75% max. |
| Chromium (Cr) | 17.00-19.00% |
| Nickel (Ni) | 13.00-15.50% |
| Molybdenum (Mo) | 2.00-3.00% |
| Nitrogen (N) | 0.10% max. |
| Copper (Cu) | 0.50% max. |
| Iron (Fe) | Balance |

The ring diameter is very small, so the tubing from which it is made must necessarily also have a small diameter. Typically the stent and rings have an outer diameter on the order of about 0.06 inch in the unexpanded condition, the same outer diameter of the tubing from which it is made, and can be expanded to an outer diameter of 0.1 inch or more. The wall thickness of the tubing is about 0.003 inch.

The tubing is mounted in a rotatable collet fixture of a machine-controlled apparatus for positioning the tubing relative to a laser. According to machine-encoded instructions, the tubing is rotated and moved longitudinally relative to the laser which is also machine controlled. The laser selectively removes the material from the tubing by ablation and a pattern is cut into the tube. The tube is therefore cut into the discrete pattern of the finished cylindrical rings.

The process of cutting a pattern for the rings into the tubing is automated except for loading and unloading the length of tubing. In one example, a CNC-opposing collet fixture for axial rotation of the length of tubing is used in conjunction with a CNC X/Y table to move the length of tubing axially relatively to a machine-controlled controlled laser. The entire space between collets can be patterned using the CO2 laser set-up of the foregoing example. The program for control of the apparatus is dependent on the particular configuration used and the pattern to be ablated in the coating.

Cutting a fine structure (0.0035 inch web width) requires minimal heat input and the ability to manipulate the tube with precision. It is also necessary to support the tube yet not allow the stent structure to distort during the cutting operation. In order to successfully achieve the desired end results, the entire system must be configured very carefully. The tubes are typically made of stainless steel with an outside diameter of 0.060 inch to 0.066 inch and a wall thickness of 0.002 inch to 0.004 inch. These tubes are fixtured under a laser and positioned utilizing a CNC to generate a very intricate and precise pattern. Due to the thin wall and the small geometry of the ring pattern (0.0035 inch typical web width), it is necessary to have very precise control of the laser, its power level, the focused spot size, and the precise positioning of the laser cutting path.

In order to minimize the heat input into the ring structure, which prevents thermal distortion, uncontrolled burn out of the metal, and metallurgical damage due to excessive heat, and thereby produce a smooth debris free cut, a Q-switched Nd-YAG, typically available from Quantronix of Hauppauge, N.Y., that is frequency doubled to produce a green beam at 532 nanometers is utilized. Q-switching produces very short pulses (<100 nS) of high peak powers (kilowatts), low energy per pulse (≦3 mJ), at high pulse rates (up to 40 kHz). The frequency doubling of the beam from 1.06 microns to 0.532 microns allows the beam to be focused to a spot size that is 2 times smaller, therefore increasing the power density by a factor of 4 times. With all of these parameters, it is possible to make smooth, narrow cuts in the stainless tubes in very fine geometries without damaging the narrow series of undulations having peaks and valleys that make up the stent structure. Hence, the system of the present invention makes it possible to adjust the laser parameters to cut narrow kerf width which will minimize the heat input into the material.

The positioning of the tubular structure requires the use of precision CNC equipment such as that manufactured and sold by Anorad Corporation. In addition, a unique rotary mechanism has been provided that allows the computer program to be written as if the pattern were being cut from a flat sheet. This allows both circular and linear interpolation to be utilized in programming. Since the finished structure of the rings is very small, a precision drive mechanism is required that supports and drives both ends of the tubular structure as it is cut. Since both ends are driven, they must be aligned and precisely synchronized, otherwise the tubular structure would twist and distort as it is being cut. After the rings 12 are cut from the tube, and depending on manufacturing convenience, the rings can be separated and individually processed, or processed while still connected in tubular form and later seperated.

The optical system which expands the original laser beam, delivers the beam through a viewing head and focuses the beam onto the surface of the tube, incorporates a coaxial gas jet and nozzle that helps to remove debris from the kerf and cools the region where the beam interacts with the material as the beam cuts and vaporizes the metal. It is also necessary to block the beam as it cuts through the top surface of the tube and prevent the beam, along with the molten metal and debris from the cut, from impinging on the opposite surface of the tube.

In addition to the laser and the CNC positioning equipment, the optical delivery system includes a beam expander to increase the laser beam diameter, a circular polarizer, typically in the form of a quarter wave plate, to eliminate polarization effects in metal cutting, provisions for a spatial filter, a binocular viewing head and focusing lens, and a coaxial gas jet that provides for the introduction of a gas stream that surrounds the focused beam and is directed along the beam axis. The coaxial gas jet nozzle (0.018 inch I.D.) is centered around the focused beam with approximately 0.010 inch between the tip of the nozzle and the tubing. The jet is pressurized with oxygen at 20 psi and is directed at the tube with the focused laser beam exiting the tip of the nozzle (0.018 inch dia.). The oxygen reacts with the metal to assist in the cutting process very similar to oxyacetylene cutting. The focused laser beam acts as an ignition source and controls the reaction of the oxygen with the metal. In this manner, it is possible to cut the material with a very fine kerf with precision. In order to prevent burning by the beam and/or molten slag on the far wall of the tube I.D., a stainless steel mandrel (approx. 0.034 inch dia.) is placed inside the tube and is allowed to roll on the bottom of the tube as the pattern is cut. This acts as a beam/debris block protecting the far wall I.D.

Alternatively, this may be accomplishedby inserting a second tube inside the ring tubing which has an opening to trap the excess beam energy that is transmitted through the kerf. This second tubing also collects the debris that is ejected from the laser cut kerf. A vacuum or positive pressure can be placed in this shielding tube to remove the collection of debris.

Another technique that could be utilized to remove the debris from the kerf and cool the surrounding material would be to use the inner beam blocking tube as an internal gas jet. By sealing one end of the tube and making a small hole in the side and placing it directly under the focused laser beam, gas pressure could be applied creating a small jet that would force the debris out of the laser cut kerf from the inside out. This would eliminate any debris from forming or collecting on the inside of the stent structure. It would place all the debris on the outside. With the use of special protective coatings, the resultant debris can be easily removed.

In most cases, the gas utilized in the jets may be reactive or non-reactive (inert). In the case of reactive gas, oxygen or compressed air is used. Compressed air is used in this application since it offers more control of the material removed and reduces the thermal effects of the material itself. Inert gas such as argon, helium, or nitrogen can be used to eliminate any oxidation of the cut material. The result is a cut edge with no oxidation, but there is usually a tail of molten material that collects along the exit side of the gas jet that must be mechanically or chemically removed after the cutting operation.

The cutting process utilizing oxygen with the finely focused green beam results in a very narrow kerf (approx. 0.0005 inch) with the molten slag re-solidifying along the cut. This traps the cut out scrap of the pattern requiring further processing. In order to remove the slag debris from the cut allowing the scrap to be removed from the remaining stent pattern, it is necessary to soak the cut tube in a solution of HCl for approximately eight minutes at a temperature of approximately 55° C. Before it is soaked, the tube is placed in a bath of alcohol/water solution and ultrasonically cleaned for approximately one minute to remove the loose debris left from the cutting operation. After soaking, the tube is then ultrasonically cleaned in the heated HCl for one to four minutes depending upon the wall thickness. To prevent cracking/breaking of the struts attached to the material left at the two ends of the ring pattern due to harmonic oscillations induced by the ultrasonic cleaner, a mandrel is placed down the center of the rings 12 during the cleaning/scrap removal process. At completion of this process, the rings 12 are rinsed in water. They are now ready for electropolishing.

The rings 12 are preferably electrochemically polished in an acidic aqueous solution such as a solution of ELECTRO-GLO#300, sold by ELECTRO-GLO Co., Inc. in Chicago, Ill., which is a mixture of sulfuric acid, carboxylic acids, phosphates, corrosion inhibitors and a biodegradable surface active agent. The bath temperature is maintained at about 110°-1350° F. and the current density is about 0.4 to about 1.5 amps per in. 2. Cathode to anode area should be at least about two to one. The stents may be further treated if desired, for example by applying a biocompatible coating.

It will be apparent that both focused laser spot size and depth of focus can be controlled by selecting beam diameter and focal length for the focusing lens. It will be apparent that increasing laser beam diameter, or reducing lens focal length, reduces spot size at the cost of depth of field.

Direct laser cutting produces edges which are essentially perpendicular to the axis of the laser cutting beam, in contrast with chemical etching and the like which produce pattern edges which are angled. Hence, the laser cutting process essentially provides stent cross-sections, from cut-to-cut, which are square or rectangular, rather than trapezoidal. The cross-sections have generally perpendicular edges formed by the laser cut. The resulting cylindrical rings 12 provide superior performance.

Other methods of forming the rings of the present invention can be used, such as chemical etching; electric discharge machining; laser cutting a flat sheet and rolling it into a cylinder; and the like, all of which are well known in the art at this time.

The tube-to-ring attachment as shown in FIG. 4 and FIG. 10 can be accomplished in many ways including slotting the polymeric tube, using a bonding agent or by interference fit. For example, when using a bonding agent, the adhesive 23 can be applied at points of convergence in the polymeric mesh 21 and at points in between the peaks and valleys in the cylindrical rings 12. The adhesive 23 can be any biocompatible adhesive that is well known, such as a cyanoacrylite-based adhesive. Several adhesives can be used including Locitite 401, 1-06FL, and M-11FL, the latter two of which are urethane-based adhesives. Other adhesives can be used without departing from the spirit and scope of the invention. As can be seen in FIG. 10, the adhesive 23 forms the bond for attaching the cylindrical rings 12 to the polymeric tube 13. After the adhesive 23 solidifies, the stent assembly is removed from the mandrel 22.

While the invention has been described in connection with certain disclosed embodiments, it is not intended to limit the scope of the invention to the particular forms set forth, but, on the contrary it is intended to cover all such alternatives, modifications, and equivalents as may be included in the spirit and scope of the invention as defined by the appended claims.

What is claimed:

1. An intravascular stent, comprising:
a plurality of metallic cylindrical rings having first and second delivery diameters;
a polymeric tube having first and second delivery diameters and an outer surface;
wherein the cylindrical rings are aligned along a longitudinal axis of the stent and attached to the outer surface of the polymeric tube.

2. The stent of claim 1, wherein longitudinal resistance to bending is at least 200% less than a metallic stent having the same size and shape.

3. The stent of claim 1, wherein radial resistance to compression is at least 200% greater than a metallic stent having the same size and shape.

4. The stent of claim 1, wherein the cylindrical rings and polymeric tube are continuously coupled together in both the first delivery diameter and second delivery diameter respectively.

5. The stent of claim 1, wherein a plurality of integral protrusions extend radially outward of the cylindrical rings in the second implanted diameter.

6. The stent of claim 1, wherein the rings are attached to the polymeric tube with a bonding agent.

7. The stent of claim 1, wherein the rings fit within slots in the outer surface of the polymeric tube.

8. The stent of claim 1, wherein the polymeric tube is formed with a mesh pattern.

9. The stent of claim 8, wherein the cylindrical rings overlap the mesh pattern.

10. The stent of claim 9, wherein less than 20% of the metallic material forming the cylindrical rings overlaps the mesh pattern.

11. The stent of claim 9, wherein less than 15% of the polymeric material forming the mesh pattern is overlapped by the cylindrical rings.

12. The stent of claim 8, wherein the mesh pattern compresses when the stent is crimped onto a catheter and expands when the stent is deployed from the catheter.

13. The stent of claim 8, wherein the mesh pattern has converging points to which the cylindrical rings are bonded.

14. The stent of claim 1, wherein the cylindrical rings have undulations comprising peaks and valleys.

15. The stent of claim 14, wherein a plurality of cylindrical rings are bonded to the polymeric tube at points in between the plurality of peaks and valleys of the cylindrical rings.

16. The stent of claim 14, wherein the peaks and valleys of a plurality of cylindrical rings form U-shaped portions.

17. The stent of claim 14, wherein the peaks and valleys of a plurality of cylindrical rings form Y-shaped portions.

18. The stent of claim 14, wherein the peaks and valleys of a plurality of cylindrical rings form W-shaped portions.

19. The stent of claim 14, wherein the peaks of each cylindrical ring are axially aligned with the valleys of each adjacent cylindrical ring.

20. The stent of claim 1, wherein the polymer material forming the tube embodies shape memory characteristics.

21. The stent of claim 1, wherein the polymeric material forming the tube is loaded with a therapeutic drug.

22. The stent of claim 1, wherein the polymeric tube is coated with a therapeutic drug.

23. The stent of claim 1, wherein a plurality of metallic cylindrical rings are coated with a therapeutic drug.

24. The stent of claim 1, wherein the stent is biodegradable.

25. The stent of claim 1, wherein the stent is non-biodegradable.

26. The stent of claim 1, wherein a material is compounded into the polymeric tube to generate a magnetic susceptibility artifact of the stent.

27. The stent of claim 1, wherein the polymeric tube includes a material therein to enhance the radiopacity of the stent.

28. The stent of claim 1, wherein the cylindrical rings include a material therein to enhance the radiopacity of the stent.

29. The stent of claim 1, wherein the stent may be expanded by force.

30. The stent of claim 1, wherein the stent is self-expanding.

31. The stent of claim 30, wherein the cylindrical rings are made from a shape memory alloy.

32. The stent of claim 31, wherein the shape memory alloy is a superelastic material.

33. The stent of claim 32, wherein the superelastic material is a nickel titanium alloy.

34. The stent of claim 1, wherein at least four cylindrical rings are attached to the polymeric tube.

35. The stent of claim 1, wherein the metallic material forming the cylindrical rings is taken from the group of alloys consisting of stainless steel, titanium, tantalum, nickel titanium, cobalt-chromium, gold, paladium, platinum and iradium.

36. The stent of claim 1, wherein the polymer material forming the polymeric tube is taken from the group of polymers consisting of polyurethanes, polyolefins, polyesters, polyamides, flouropolymers and their co-polymers, polyetherurethanes, polyesterurethanes, silicone, thermoplastic elastomer (e.g., C-flex), polyether-amide thermoplastic elastomer (e.g., Pebax), fluoroelastomers, fluorosilicone elastomer, styrene-butadiene-styrene rubber, styrene-isoprene-styrene rubber, polyisoprene, neoprene (polychloroprene), polybutadienne-ethylene-propylene elastomer, chlorosulfonated polyethylene elastomer, butyl rubber, polysulfide elastomer, polyacrylate elastomer, nitrile rubber, a family of elastomers composed of styrene, ethylene, propylene, aliphatic polycarbonate polyurethane, polymers augmented with antioxidents, polymers augmented with image enhancing materials, polymers having a proton (H+) core, polymers augmented with protons (H+), butadiene and isoprene (e.g., Kraton) and polyester thermoplastic elastomer (e.g., Hytrel).

37. A method for forming an intravascular stent, comprising:
fitting a plurality of outer mold covers around a mandrel;
injecting a polymer into the outer mold covers to form a polymeric tube;
removing the outer mold covers;
forming a plurality of metallic cylindrical rings; and
fitting the plurality of metallic cylindrical rings over the polymeric tube.

38. A method for forming an intravascular stent, comprising:
means for forming a polymeric tube;
means for forming a plurality of cylindrical rings; and
means for securing the cylindrical rings on an outer surface of the polymeric tube.

39. The method of claim 38, wherein the means for forming the cylindrical rings comprise laser cutting the rings.

40. The method of claim 38, wherein the means for forming a polymeric tube comprise injection molding the tube.

41. The method of claim 38, wherein means for securing the cylindrical rings on the outer surface of the polymeric tube includes bonding the cylindrical rings to the outer surface of the polymeric tube.
